(19) United States
(12) Patent Application Publication
(10) Pub. No.: US 2025/0222235 A1
(43) Pub. Date: Jul. 10, 2025

(54) IMPROVED URETERAL ACCESS SHEATH

(71) Applicant: ZHEJIANG YIGAO MEDICAL TECHNOLOGY CO., LTD., Zhejiang (CN)

(72) Inventors: FANGBING LI, Zhejiang (CN); Xing LI, Zhejiang (CN)

(73) Assignee: ZHEJIANG YIGAO MEDICAL TECHNOLOGY CO., LTD., Zhejiang (CN)

(21) Appl. No.: 18/879,224

(22) PCT Filed: Sep. 13, 2022
(86) PCT No.: PCT/CN2022/118402

§ 371 Date: Dec. 26, 2024

(30) Foreign Application Priority Data
Dec. 24, 2021 (CN) 202111601835.9

(51) Int. Cl. A61M 25/06; A61M 25/00; A61M 29/00

(52) U.S. Cl. CPC A61M 25/0662; A61M 25/0054; A61M 29/00; A61M 2025/0681; A61M 2205/0216; A61M 2205/3344; A61M 2210/1078

(57) ABSTRACT

An improved ureteral sheath, includes: an introducer sheath including a sheath tube and a sheath base arranged at a proximal end of the sheath tube, the sheath base is provided with a tube segment in communication with the sheath tube and a connecting part arranged at a distal end of the tube segment; the sheath tube is provided, from a proximal end to a distal end thereof, with a main body segment and a head segment arranged at a distal end of the main body segment, the main body segment is composed of a hard tube segment, and the head segment is composed of a deformable tube segment; and the tube segment extends outwards relative to a central axis thereof in a communication manner to form a negative pressure connecting tube, a tube wall of the negative pressure connecting tube is provided with vent groove.

### TECHNICAL FIELD

The disclosure belongs to the field of urological medical instruments, in particular to an improved ureteral access sheath.

### BACKGROUND

A ureteral access sheath is used in urological surgery to establish a convenient access for endoscopes and other instruments to enter the urinary tract. In order to improve the service life of the ureteral access sheath and lumen support, spiral steel wires are embedded in walls of the ureteral access sheath, so that the ureteral access sheath has relatively high elasticity and certain hardness, cannot be bent and shaped in a short distance, and the end of the ureteral access sheath cannot approach a lesion area at close range. During surgical operations, the expulsion of blood clots, small stones, and purulent flocculent matter through the ureteral access sheath is less effective; the end of the ureteral access sheath is too hard to bend, which prevents close access to the lesion area and increases the difficulty and time of operation.

In addition, an introducer sheath also has a negative pressure suction function, so the endoscope needs to be sealed and matched with the introducer sheath; when the existing endoscope uses a sealing valve to seal and match with the introducer sheath, since a dilator needs to be inserted or pulled out, there is interference between the existing sealing valve and the installation position of the dilator. As a result, doctors must install the sealing valve after removing the dilator and then insert the endoscope, and the endoscope contacts the sealing valve surface, which easily causes difficulty in inserting the endoscope and making the procedure complex and laborious for doctors.

In addition, the existing ureteral access sheath installation structure is complex and the negative pressure suction function cannot be adjusted.

### SUMMARY OF INVENTION

Based on the above problems, the present disclosure provides an improved ureteral access sheath, the ureteral access sheath includes:
an introducer sheath, comprising a sheath tube and a sheath base mounted on a proximal end of the sheath tube, the sheath base having a tube segment communicating with the sheath tube and a connecting part arranged at a distal end of the tube segment; wherein the connecting part includes at least two snap-fit elements arranged on the distal end of the tube segment, which are bent towards the inside with respect to a central axis of the tube segment; the sheath tube and the tube segment integrally define a central passage extending from the proximal end to the distal end; the sheath tube has a main body segment from the proximal end toward the distal end and a head segment arranged at the distal end of the main body segment, and the main body segment is formed by a hard tube segment and the head segment is formed by a deformable tube segment; the tube segment communicates and extends outward relative to its central axis to form a negative pressure connecting tube, a tube wall of the negative pressure connecting tube is provided with an open vent groove, an upper sealing cover of the vent groove is provided with a sliding cover, and the sliding cover can slide relative to the vent groove, and by sliding the sliding cover, the opening area of the vent groove can be adjusted, thereby adjusting the negative pressure;
a dilator, including a catheter and a dilator base mounted at the proximal end of the catheter, wherein the catheter is arranged through the central passage, and the dilator base includes a base body and at least two elastic hangers symmetrically arranged on the base body, and the base body is connected with the distal end of the catheter, and the elastic hangers extend outward relative to the base body, and the elastic hangers can be in snap connection with the snap-fit element;
a tapered sealing ring, arranged at the tube segment of the sheath base, the tapered sealing ring forming an inlet for passage of the catheter to seal therethrough, ribs formed on an inner wall of the tapered sealing ring;
a gland, fixedly installed at the tube segment of the sheath base and having an opening for passage of the catheter, wherein at least the upper edge of the tapered sealing ring is connected to the gland.

Preferably, the base body is a tube base communicating with the catheter, and the tube base extends outward relative to its central axis, with two elastic arms provided, and the tail ends of the elastic arms form hangers.

Preferably, a cross-sectional area of the tube segment gradually decreases from the proximal end to the distal end; two limiting platforms are symmetrically arranged on the upper side of the tube segment, and the elastic hanger can abut against the limiting platform.

Preferably, the vent groove is arranged to extend along the length of the tube wall, and the sliding cover is clipped on an inner edge of the vent groove through a sliding part; two fixture blocks are symmetrically arranged on the left and right inside the sliding cover, the fixture blocks extend into the vent groove and prop against the inner sides of left and right edges of the vent groove respectively, and the fixture blocks can slide linearly along the two edges of the vent groove.

Preferably, the tapered sealing ring includes a tapered wall defining a tapered passage, the tapered wall has a proximally located first annular edge and a distally located second annular edge, an inner wall of the tapered wall is circumferentially spaced with ribs extending from the first annular edge toward the second annular edge.

Preferably, the gland includes a gland body and a tapered ring pressed against the inner wall of an inlet side of the tapered wall.

Preferably, the tapered sealing ring is a rubber ring, and the second annular edge of the rubber ring defines an aperture capable of sealingly engaging the catheter of the dilator.

Preferably, the deformable tube segment is a hose.

Preferably, the length of the head segment is configured to match the longitudinal length of a bending part of an endoscope.

Preferably, the hard tube segment is integrally molded or adhesively connected with the deformable tube segment.

With the ureteral access sheath provided by the present disclosure, the elastic hangers of the dilator base are clipped in the snap-fit element of the sheath base, and the sheath tube can be taken out by rotating left and right or pinching left and right hangers inward, which is convenient for introduction and operation.

By improving the sealing valve of the sheath tube, the sealing of the negative pressure passage in the sheath tube can be ensured, and the design of the ribs can reduce the tension of the sealing valve on an insertion instrument and reduce the resistance when a user inserts the instrument.

With the ureteral access sheath provided by the present disclosure, it is possible to manually slide the sliding cover to adjust the area of an open-air vent groove, thereby adjusting the negative pressure suction pressure.

With the ureteral access sheath provided by the present disclosure, it can be ensured that the head segment of the ureteral access sheath can bend along with the bending part of the endoscope. In this way, the ureteral access sheath can enter into the depth of a renal pelvis at a negative angle to a ureter, and can efficiently and thoroughly suck up stones, thus reducing the difficulty and time of surgery.

### BRIEF DESCRIPTION OF DRAWINGS

FIG. 1 is a structural schematic diagram of the ureteral access sheath provided by the present disclosure;

FIG. 2 is a structural sectional diagram of the ureteral access sheath provided by the present disclosure;

FIG. 3 is a structural schematic diagram of the sealing valve provided by the present disclosure;

FIG. 4 is a structural schematic diagram of the introducer sheath provided by the present disclosure.

### DESCRIPTION OF EMBODIMENTS

The technical scheme of the present disclosure will be further described in detail below through specific embodiments and in combination with drawings of the specification;

It should be understood in the description of the present disclosure that the directions or position relationships such as “front”, “rear”, “head”, “tail”, “distal”, “proximal”, “axial” and “radial” are based on those shown in the accompanying drawings, and are used only for facilitating the description of the present disclosure and simplifying the description, not for indicating or implying that the target devices or elements must have a special direction and be constructed and operated at the special direction, so they cannot be understood as the restrictions to the present disclosure.

In the description of the present disclosure, it should be noted that the terms “mount” and “connect” should be understood in a broad sense unless otherwise specified and defined. For example, it may refer to mechanical connection or electrical connection, and may also refer to internal connection or direct connection of two elements or indirect connection of two elements by an intermedium. The person of ordinary skill in the art can understand the specific meaning of the above terms according to specific conditions.

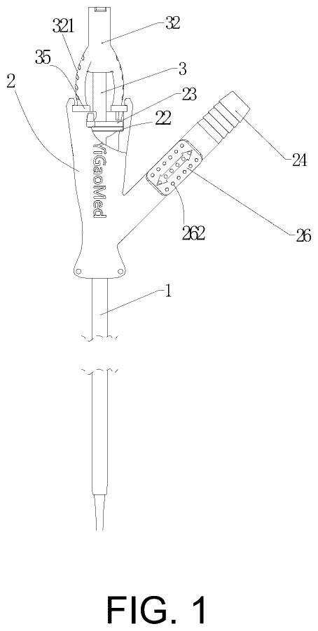

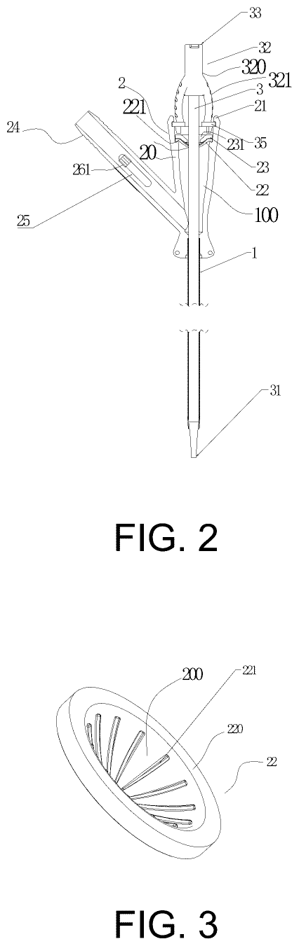

Please refer to FIG. 1 and FIG. 2. The embodiments of the present disclosure provide a ureteral access sheath with an improved installation structure, the ureteral access sheath including an introducer sheath and a dilator 3. By improving the installation structure, the dilator can be better assembled with the introducer sheath in a detachable manner; specifically, the ureteral access sheath provided by this embodiment includes the following structures:

The introducer sheath includes a sheath tube 1 and a sheath base mounted on a proximal end of the sheath tube, the sheath base 2 having a tube segment 20 communicating with the sheath tube and a connecting part 21 arranged at a distal end of the tube segment; the connecting part 21 includes at least two snap-fit elements 21 arranged on the distal end of the tube segment, and the snap-fit elements 21 are bent towards the inside with respect to a central axis of the tube segment 20; the sheath tube 1 and the tube segment 20 integrally define a central passage 100 extending from the proximal end to the distal end;

The dilator 3 includes a catheter 31 and a dilator base 32 mounted at the proximal end of the catheter 31 and the catheter 31 is arranged through the central passage 100, and the dilator base 32 includes a base body 320 and at least two elastic hangers 35 symmetrically arranged on the base body 320, and the base body 320 is connected with the distal end of the catheter 31, and the elastic hangers 35 extend outward relative to the base body 320, and the elastic hangers 35 can be in snap connection with the snap-fit element 21; in some embodiments, the base body 320 is a tube base communicating with the catheter 31, and the tube base extends outward relative to its central axis, with two elastic arms 321 provided, and the tail ends of the elastic arms form hangers 35; specifically, Luer connectors 33 are connected to the tube base. In addition, in the embodiment, the cross-sectional area of the tube segment 20 gradually decreases from the proximal end to the distal end so as to facilitate dilator access. In the embodiment, two limiting platforms 21 are symmetrically arranged on the upper side of the tube segment 20, and the two limiting platforms constitute a snap-fit element, and the elastic hanger 35 can abut against the limiting platform 21. In this way, the elastic hangers 35 of the dilator can be fixed on the limiting platform, and the dilator can be taken out by rotating left and right or pinching left and right hangers inward. The whole process is easy to operate and convenient for doctors to apply force. Compared with the prior art that the dilator base is mounted outside an introducer sheath base, the dilator base provided in the present disclosure is mounted inside the introducer sheath, which can be taken out instantaneously by rotating or pinching inward, and mounting inward makes the operation of the dilator easier and allows for a smoother, more effortless operation.

The ureteral access sheath provided in the embodiment also includes a tapered sealing ring 22 and a gland 23. The tapered sealing ring 22 is arranged in the tube segment 20 of the sheath base 2, the tapered sealing ring 22 forming an inlet 220 for the catheter to seal therethrough. It can be seen from FIG. 3 that multiple ribs 221 are formed on the inner wall of the tapered sealing ring 22 and the gland 23 is fixedly mounted in the tube segment of the sheath base 2 and has an opening 231 through which the catheter 31 passes, and the upper edge of the tapered sealing ring is connected with the gland; that is, compared with the existing sealing valve, the sealing valve provided in the embodiment is directly mounted at the valve base, which eliminates the user's operation of mounting the sealing valve and is easy to operate; the ribs are designed to reduce the tension of the sealing valve on the insertion instruments, thus reducing the resistance when the user inserts instruments. Specifically, the tapered sealing ring includes a tapered wall defining a tapered passage, the tapered wall having a proximally located first annular edge 220 and a distally located second annular edge 222, an inner wall of the tapered wall being circumferentially spaced with a plurality of ribs 221 extending from the first annular edge 220 toward the second annular edge 222; such that as the catheter of the dilator passes through the sheath, linear friction is formed with the sealing valve, eliminating the original surface contact and saving effort when pushing in. In this embodiment, the gland 23 includes a gland body and a tapered ring pressed against the inner wall of an inlet side of the tapered wall, so that the sealing valve can be better supported to avoid eversion of the sealing valve when the dilator enters or exits from the sheath tube. Specifically, the tapered sealing ring is a rubber ring, and it can be understood that the second annular edge of the rubber ring defines an aperture capable of sealingly engaging the catheter of the dilator. By improving the sealing valve of the sheath tube, the sealing of the negative pressure passage in the sheath tube can be ensured, and the design of the ribs can reduce the sealing valve on an insertion instrument whose a tube wall of the connecting tube 24 being provided with an open vent groove 25, an upper sealing cover of the vent groove 25 is provided with a sliding cover 26, and the sliding cover 26 can slide relative to the vent groove 25, and by sliding the sliding cover 26, the opening area of the vent groove 25 can be adjusted, thereby adjusting the negative pressure; when the negative pressure connecting tube is connected with a suction machine, the suction pressure is fixed and can be adjusted by sliding the sliding cover; the disclosure solves the tedious problem of manual plugging, with convenient and rapid operation, improved efficiency, and stable and controllable pressure adjustment process; in some preferred embodiments, the vent groove 25 is arranged to extend along the length of the tube wall, and the sliding cover is clipped on an inner edge of the vent groove 251 through a sliding part. Specifically, two fixture blocks 261, 262 are symmetrically arranged on the left and right inside the sliding cover 26, the fixture blocks 261, 262 extending into the vent groove and propping against the inner sides of left and right edges 251 of the vent groove respectively, and the fixture blocks 261, 262 can slide linearly along the two edges of the vent groove 25. In this way, the opening area of the vent groove can be adjusted to adjust the suction pressure. In the embodiment, the outer surface of the sliding cover 26 has a rough surface 260 to increase manual friction force, avoid hand slipping and facilitate operation; optionally, the rough surface is composed of skid-proof stripes; in the embodiment, the connecting part 231 includes at least two snap-fit elements arranged on the distal end of the tube segment, and the snap-fit elements are bent towards the inside with respect to the central axis of the tube segment; the connecting part may also be realized in other forms; through the ureteral access sheath provided by the present disclosure, the area of the open-air vent groove can be adjusted by manually sliding the sliding cover, so as to adjust the negative pressure suction pressure.

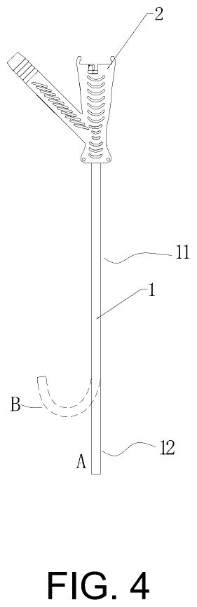

Referring to FIG. 4, the present embodiment provides the introducer sheath that can be used in conjunction with an endoscope, and the sheath tube 1 has a main body segment 11 extending from the proximal end to the distal end, and a head segment 12 positioned at the distal end of the main body segment 11, and the main body segment 11 is composed of a hard tube segment, and the head segment 12 is made of a deformable tube segment, so that the bending part of the endoscope extending into the deformable tube segment can drive the deformable tube to bend and change from a vertical state A to a bending state B; this allows access to the renal pelvis through the ureteral curvature. In some embodiments, the deformable tube segment is a hose; specifically, the length of the head segment 12 is set to match the longitudinal length of the endoscope bending part; in the embodiment, the hard tube segment 11 and the deformable tube segment 12 are integrally molded or adhesively connected. The endoscope is an existing device, which includes an operating part and a bending part, and the operating part can drive the bending part to bend; through the introducer sheath provided by the disclosure, it can be ensured that the head segment of the ureteral access sheath can bend with the bending part of the endoscope, so that the head segment of the sheath tube can enter into the depth of a renal pelvis at a negative angle to a ureter, and stones can be efficiently and thoroughly sucked up, thus reducing the difficulty and time of surgery.

In the description of the specification, descriptions with reference to the terms “one embodiment”, “some embodiments”, “example”, “specific example”, or “some examples” mean that specific features, structures, materials or characteristics described in combination with the embodiment or example are contained in at least one embodiment or example of the present disclosure. In the specification, the schematic expressions of the above terms do not necessarily refer to the same embodiments or examples. Moreover, the specific features, structures, materials, or characteristics described may be combined in any one or more embodiments or examples in a suitable manner.

Although embodiments of the present disclosure have been shown and described, the person of ordinary skill in the art can understand that various changes, modifications, substitutions and variations may be made to these embodiments without departing from the principle and purpose of the present disclosure, and the scope of the present disclosure is defined by the claims and their equivalents.

1. An improved ureteral access sheath, wherein the ureteral access sheath comprises:
an introducer sheath, comprising a sheath tube and a sheath base mounted on a proximal end of the sheath tube, the sheath base having a tube segment communicating with the sheath tube and a connecting part arranged at a distal end of the tube segment; wherein the connecting part comprises at least two snap-fit elements arranged on the distal end of the tube segment, which are bent towards an inside with respect to a central axis of the tube segment; the sheath tube and the tube segment integrally define a central passage extending from the proximal end to the distal end; the sheath tube has a main body segment from the proximal end toward the distal end and a head segment arranged at a distal end of the main body segment, and the main body segment is formed by a hard tube segment and the head segment is formed by a deformable tube segment; the tube segment communicates and extends outward relative to the central axis to form a negative pressure connecting tube, a tube wall of the negative pressure connecting tube is provided with a vent groove which is opened, an upper sealing cover of the vent groove is provided with a sliding cover, and the sliding cover is slidable relative to the vent groove, and by sliding the sliding cover, an opening area of the vent groove is adjustable, thereby adjusting negative pressure;
a dilator, comprising a catheter and a dilator base mounted at a proximal end of the catheter, and the catheter is arranged through the central passage, and the dilator base comprises a base body and at least two elastic hangers symmetrically arranged on the base body, and the base body is connected with a distal end of the catheter, and the elastic hangers extend outward relative to the base body, and the elastic hangers is able to be in snap connection with the snap-fit elements;
a tapered sealing ring, arranged at the tube segment of the sheath base, the tapered sealing ring forming an inlet for passage of the catheter to seal therethrough, ribs formed on an inner wall of the tapered sealing ring;
a gland, fixedly installed at the tube segment of the sheath base and having an opening for passage of the catheter, at least an upper edge of the tapered sealing ring is connected to the gland.

2. The ureteral access sheath according to claim 1, wherein the base body is a tube base communicating with the catheter, and the tube base extends outward relative to its central axis, with two elastic arms provided, and tail ends of the elastic arms form hangers.

3. The ureteral access sheath according to claim 1, wherein a cross-sectional area of the tube segment gradually decreases from the proximal end to the distal end; two limiting platforms are symmetrically arranged on an upper side of the tube segment, and the elastic hangers are able to abut against the limiting platforms.

4. The ureteral access sheath according to claim 1, wherein the vent groove is arranged to extend along a length of the tube wall, and the sliding cover is clipped on an inner edge of the vent groove through a sliding part; two fixture blocks are symmetrically arranged on left and right inside the sliding cover, the fixture blocks extend into the vent groove and prop against inner sides of left and right edges of the vent groove respectively, and the fixture blocks is able to slide linearly along the left and right edges of the vent groove.

5. The ureteral access sheath of claim 1, wherein the tapered sealing ring comprises a tapered wall defining a tapered passage, the tapered wall has a first annular edge which is located proximally and a second annular edge which is located distally, an inner wall of the tapered wall is circumferentially spaced with a plurality of ribs extending from the first annular edge toward the second annular edge.

6. The ureteral access sheath according to claim 5, wherein the gland comprises a gland body and a tapered ring pressed against the inner wall of an inlet side of the tapered wall.

7. The ureteral access sheath according to claim 6, wherein the tapered sealing ring is a rubber ring, and the second annular edge of the rubber ring defines an aperture capable of sealingly engaging the catheter of the dilator.

8. The ureteral access sheath according to claim 6, wherein the deformable tube segment is a hose.

9. The ureteral access sheath according to claim 1, wherein a length of the head segment is configured to match a longitudinal length of a bending part of an endoscope.

10. The ureteral access sheath according to claim 1, wherein the hard tube segment is integrally molded or adhesively connected with the deformable tube segment.
